(19) United States
(12) Patent Application Publication
(10) Pub. No.: US 2026/0115475 A1
(43) Pub. Date: Apr. 30, 2026

(54) CLOSED-LOOP NEUROMODULATION SYSTEM BASED ON SACRAL NERVE ELECTROPHYSIOLOGICAL ACTIVITY MONITORING

(71) Applicants: CHINA REHABILITATION SCIENCE INSTITUTE, Beijing (CN); CHINA REHABILITATION RESEARCH CENTER, Beijing (CN)

(72) Inventor: Limin LIAO, Beijing (CN)

(73) Assignees: CHINA REHABILITATION SCIENCE INSTITUTE, Beijing (CN); CHINA REHABILITATION RESEARCH CENTER, Beijing (CN)

(21) Appl. No.: 19/337,890

(22) Filed: Sep. 23, 2025

(30) Foreign Application Priority Data
Oct. 30, 2024 (CN) 202411523993.0

(51) Int. Cl. A61N 1/36; A61N 1/02; A61N 1/372; G16H 20/40; G16H 40/60

(52) U.S. Cl. CPC A61N 1/36139; A61N 1/025; A61N 1/36007; A61N 1/37247; G16H 20/40; G16H 40/60

(57) ABSTRACT

The present disclosure relates to a closed-loop neuromodulation system based on sacral nerve electrophysiological activity monitoring. The closed-loop neuromodulation system includes an implantable neuromodulation module, a programming module, and a server module. The implantable neuromodulation module obtains monitoring data based on an implantable electrode and synchronously obtains a local field potential variation signal generated in a patient with urinary tract dysfunction during the sacral nerve conduction, and applies an electrical stimulation pulse to the sacral nerve of the patient when an abnormal monitoring result of nerve afferent impulses associated with urinary urgency symptom of the patient is obtained based on the local field potential variation signal. The server module stores the monitoring data and sends an intelligent recognition model that is pre-trained to the implantable neuromodulation module and the programming module, respectively.

### CROSS-REFERENCE TO RELATED APPLICATIONS

This application claims priority to Chinese application No. 202411523993.0, filed on Oct. 30, 2024, the entire contents of which are incorporated herein by reference.

### TECHNICAL FIELD

The present disclosure relates to the field of neural electrical stimulation technology, and in particular, to a closed-loop neuromodulation system based on sacral nerve electrophysiological activity monitoring.

### BACKGROUND

Sacral neuromodulation (SNM) is a neuromodulation technology that employs an implantable electrical stimulation pulse generator to continuously apply low-frequency electrical pulses to specific sacral nerves (third sacral nerve (S3) and fourth sacral nerve (S4)) via an implantable electrode to excite or inhibit a neural pathway, modulate abnormal reflex arcs of the sacral nerve, and then affect and modulate functions of sacral nerve-innervated target organs (e.g., the bladder, urethral/anal sphincter, pelvic floor, etc.), thus achieving a treatment effect. In the related technologies, the SNM system lacks real-time responsiveness, autonomous modulation capabilities, and demonstrates suboptimal efficacy of manual intervention.

### SUMMARY

The embodiments of the present disclosure provide a closed-loop neuromodulation system based on sacral nerve electrophysiological activity monitoring, which enables the technical effects of real-time monitoring and automatic recognition of an abnormal reflex signal of the sacral nerve, and automatic application of electrical stimulation for intervention, thereby forming a complete closed-loop neuromodulation and improving the effectiveness of treatment.

The embodiments of the present disclosure employ the following technical solution.

A closed-loop neuromodulation system based on sacral nerve electrophysiological activity monitoring is provided. The system may include an implantable neuromodulation module, a programming module, and a server module. The implantable neuromodulation module may be connected with the programming module, and the server module may be connected with the programming module. The implantable neuromodulation module may be configured to obtain monitoring data based on an implantable electrode and synchronously obtain a local field potential variation signal of a patient with urinary tract dysfunction generated during a sacral nerve conduction, and the local field potential variation signal may be caused by a neural activity leading to the urinary tract dysfunction.

The implantable neuromodulation module may be configured to apply an electrical stimulation pulse to a sacral nerve of the patient when an abnormal monitoring result of nerve afferent impulses associated with urinary urgency symptom of the patient is obtained based on the local field potential variation signal, and a control signal of the electrical stimulation pulse may be generated by the programming module. The implantable neuromodulation module may also be configured to deactivate, in response to recognizing the normal monitoring result, the electrical stimulation pulse applied to the sacral nerve after a preset delay time when recognizing that the abnormal monitoring result returns to a normal monitoring result during an asymptomatic phase of the patient. The server module may be configured to store the monitoring data and send an intelligent recognition model that is pre-trained to the implantable neuromodulation module and the programming module, respectively.

The implantable neuromodulation module may include an intelligent recognition unit, and the intelligent recognition unit may be configured to identify, in real time, based on the intelligent recognition model, a first feature parameter corresponding to the local field potential variation signal generated by the patient with urinary tract dysfunction during the sacral nerve conduction. The first feature parameter may include at least one of a potential intensity variation or a potential frequency variation. The intelligent recognition unit may also be configured to determine a first monitoring result corresponding to the local field potential variation signal based on the first feature parameter and initiate the electrical stimulation pulse to the sacral nerve when the abnormal monitoring result of the nerve afferent impulses associated with the urinary urgency symptom of the patient exists.

### BRIEF DESCRIPTION OF THE DRAWINGS

The present disclosure is further illustrated in terms of exemplary embodiments. These exemplary embodiments are described in detail with reference to the drawings. These embodiments are non-limiting exemplary embodiments, in which like reference numerals represent similar structures, and wherein:

FIG. 1 is a schematic diagram illustrating a closed-loop neuromodulation system based on sacral nerve electrophysiological activity monitoring according to some embodiments of the present disclosure;

FIG. 2 is a diagram illustrating a potential signal change during a urinary urgency phase and a non-urinary urgency phase of a patient after electrode implantation according to some embodiments of the present disclosure; and

FIG. 3 is a schematic diagram illustrating an exemplary prediction model according to some embodiments of the present disclosure.

Description of markers in the accompanying drawings:

1—implantable neuromodulation module; 2—programming module; 3—server module; 11—electrode channel; 12—sacral nerve signal acquisition unit; 13—first signal processing unit; 14—first feature extraction unit; 15—intelligent recognition unit; 16—neurostimulation unit; 21—second signal processing unit; 22—second feature extraction unit; 23—display unit; 24—parameter configuration unit; 25—algorithm configuration unit; 4—pressure monitoring module; 5—prediction model; 51—bioelectrical signal; 52—intravesical pressure data; 53—patient information; 54—environmental data; 55—application time point of electrical stimulation pulse.

### DETAILED DESCRIPTION

To make the purpose, technical solutions, and advantages of the present disclosure clearer, the technical solutions of the present disclosure will be described clearly and completely in the following in conjunction with the specific embodiments of the present disclosure and the corresponding accompanying drawings. Obviously, the embodiments described are only a part of the embodiments of the present disclosure, and not all the embodiments. Based on the embodiments in the present disclosure, all other embodiments obtained by a person of ordinary skill in the art without making creative labor fall within the scope of protection of the present disclosure.

It should be understood that “system”, “device”, “unit” and/or “module” as used herein is a manner used to distinguish different components, elements, parts, sections, or assemblies at different levels. However, if other words serve the same purpose, the words may be replaced by other expressions.

As shown in the present disclosure and claims, the words “one”, “a”, “a kind” and/or “the” are not especially singular but may include the plural unless the context expressly suggests otherwise. In general, the terms “comprise”, “comprises”, “comprising”, “include”, “includes”, and/or “including”, merely prompt to include operations and elements that have been clearly identified, and these operations and elements do not constitute an exclusive listing. The methods or devices may also include other operations or elements.

The flowcharts used in the present disclosure illustrate operations that systems implement according to some embodiments of the present disclosure. It should be understood that the previous or subsequent operations may not be accurately implemented in order. Instead, each step may be processed in reverse order or simultaneously. Meanwhile, other operations may also be added to these processes, or a certain step or several steps may be removed from these processes.

To facilitate the understanding of the technical solution of a closed-loop neuromodulation system based on sacral nerve electrophysiological activity monitoring of the embodiments of the present disclosure, the technical principles of the embodiments of the present disclosure are first explained.

SNM may be applied to a variety of refractory lower urinary tract dysfunctions, including refractory overactive bladder, idiopathic urinary retention, defecation dysfunction, etc. Although the neuromodulation effect of SNM on the neural pathways has been clinically recognized, the mechanism of SNM remains to be further explored. In particular, the correlation between the signals of the sacral nerve pathways and the disease during the symptomatic phase of the patient warrants further investigation. For example, key issues still need to be addressed, such as whether specificity exists in a local electric field potential change generated by abnormal sacral nerve reflex on the neural pathway, whether a strong correlation exists between the specificity and the symptomatic phase, and whether the degree of field potential changes is associated with a conversion rate from stage I trial stimulation to stage II permanent implantation of the sacral nerve. Once a relationship between the local electric field potential change generated by the sacral nerve reflex in the symptomatic phase and the lower urinary tract dysfunction is found, and an electrophysiological biomarker-based research paradigm is established, a theoretical foundation can be laid for further improving the effectiveness of treatment.

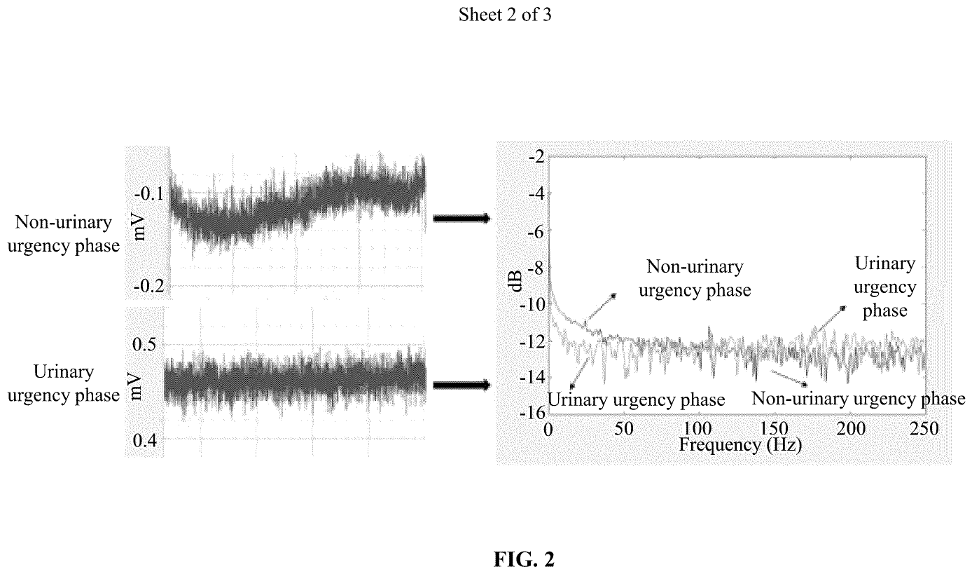

During a urinary urgency symptom of a patient, a related nerve afferent impulse is generated, which is then transmitted from the peripheral nerve to the sacral nerve root. Based on this, the present disclosure deeply studies a correlation between the urinary urgency symptom of the patient and the sacral nerve afferent impulses, and the local field potential changes. Referring to FIG. 2, the present disclosure provides a diagram illustrating a potential signal change during a urinary urgency phase and a non-urinary urgency phase of the patient after electrode implantation. The left panel of FIG. 2 is an original potential signal acquired, with a horizontal axis indicating time, and a vertical axis indicating voltage (with a unit of mV). The right panel of FIG. 2 is a spectrum analysis diagram corresponding to the original potential signal, with a horizontal axis indicating frequency (with a unit of Hz), and a vertical axis indicating signal intensity (with a unit of dB).

After stage I sacral neuromodulation electrode implantation for the patient, the electrode is placed in S3 foramen in a location adjacent to the S3 sacral nerve. The main technical scheme of the present disclosure includes acquiring potential signal data of the patient in the urinary urgency phase and the non-urinary urgency phase via the electrode and performing filtering processing and spectral transformation analysis on the potential signal data. The results of the present disclosure indicate significant differences in a spectral distribution of sacral nerve electrical signals of the patient during the non-urinary urgency phase and the urinary urgency phase. In a low-frequency range, such as a range within 1 Hz-40 Hz, the signal intensity during the non-urinary urgency phase is higher than the signal intensity during the urinary urgency phase. In a relatively high frequency range, such as a range within 100 Hz-200 Hz, the signal intensity during the non-urinary urgency phase is slightly lower than the signal intensity during the urinary urgency phase.

Therefore, the technical concept of the present disclosure is to obtain monitoring data generated during a sacral nerve conduction based on an implantable electrode, and monitor a potential change signal of the patient with urinary tract dysfunction generated by the sacral nerve in real time during the urinary urgency phase and the non-urinary urgency phase, analyze and identify an abnormal monitoring result of the nerve afferent impulses related to the urinary urgency symptom of the patient based on the correlation between the specificity of the local field potential change of the sacral nerve and the symptomatic phase of the patient and apply electrical stimulation automatically for intervention. Thus, complete closed-loop neuromodulation is formed to realize on-demand acquisition and quantitative and precise stimulation based on individualized electrophysiological biomarkers, thereby improving the treatment effect.

The following is a detailed description of the technical solutions provided by various embodiments of the present disclosure, in conjunction with the accompanying drawings.

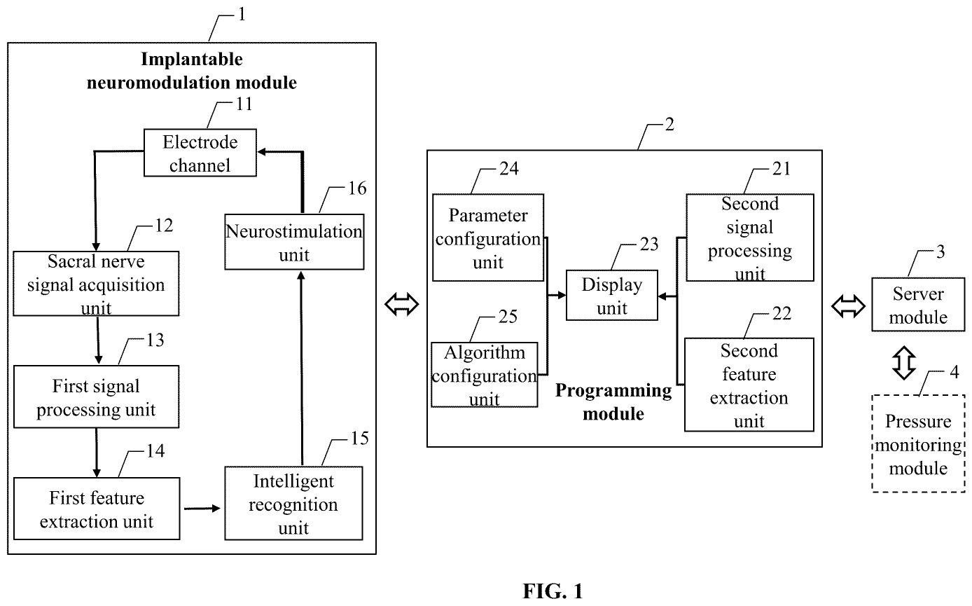

The embodiments of the present disclosure provide a closed-loop neuromodulation system based on sacral nerve electrophysiological activity monitoring, as shown in FIG. 1, a schematic diagram illustrating a structure of the closed-loop neuromodulation system based on sacral nerve electrophysiological activity monitoring is provided.

The closed-loop neuromodulation system based on sacral nerve electrophysiological activity monitoring, according to some embodiments of the present disclosure, includes an implantable neuromodulation module 1, a programming module 2, and a server module 3. The implantable neuromodulation module 1 is connected with the programming module 2, and the server module 3 is connected with the programming module 2.

In some embodiments, the implantable neuromodulation module 1, the programming module 2, and the server module 3 are all configured with communication units. For example, the implantable neuromodulation module 1 communicates with the programming module 2 wirelessly, and the programming module 2 communicates with the server module 3 via the internet, thereby minimizing restrictions on the activities of the patient. In some embodiments, the closed-loop neuromodulation system based on sacral nerve electrophysiological activity monitoring may also communicate by other wired or wireless means, which are not limited herein.

The implantable neuromodulation module 1 refers to an active module that implants a microelectronic device into the body and employs electrical stimulation or drugs to modulate the activity of the central nervous system, peripheral nerves, or autonomic nervous system. The implantable neuromodulation module 1 is usually composed of an implantable pulse generator, a lead electrode assembly, and an external programming device or implantable telemetry module. The implantable neuromodulation module 1 transmits a precisely controlled electrical pulse to a specific neuroanatomical region to modulate neural activation patterns, thereby achieving effective modulation and improvement of disease symptoms.

The implantable neuromodulation module 1 is commonly configured for deep brain stimulation (DBS), spinal cord stimulation (SCS), vagus nerve stimulation (VNS), and sacral nerve stimulation (SNS). SNS is primarily used to treat bladder dysfunction and chronic pelvic pain. Functions of the bladder and pelvic organs are modulated by implanting an electrode near the sacral nerves and delivering an electrical pulse to the sacral nerves.

In some embodiments, the implantable neuromodulation module 1 may operate offline or in conjunction with the programming module 2.

In some embodiments, when the implantable neuromodulation module 1 is operating offline, the system pre-stores standardized treatment parameters corresponding to the relevant symptoms in the implantable neuromodulation module 1, and the electrical stimulation pulses of the sacral nerve are modulated based on pre-stored standardized treatment parameters. The standardized treatment parameters may include a stimulation frequency, a pulse width, a voltage amplitude, a stimulation cycle, an electrode position, or the like.

For example, the stimulation frequency may be in a range of 10 Hz-50 Hz, the pulse width may be in a range of 100 microseconds-500 microseconds, the voltage amplitude may be in a range of 0.5 volts-10 volts, the stimulation cycle may include continuous and intermittent modes, and the electrode position may include S3 or S4 sacral foramen.

In some embodiments, when the implantable neuromodulation module 1 operates in conjunction with the programming module 2, treatment parameters input by a physician may be transmitted to the implantable neuromodulation module 1 via the programming module 2, and quantitative and precise stimulation can be achieved thereby, which is more personalized and intelligent, further enhancing the treatment capability and treatment effect.

More descriptions regarding the programming module 2 may be found in the related descriptions below and the related sections.

The implantable neuromodulation module 1 in the embodiments of the present disclosure may operate offline or in conjunction with the programming module 2, which is not only capable of maintaining the system in a stable working state without external intervention but also capable of adjusting electrical stimulation pulse parameters in real time based on a feedback and symptom change of the patient, achieving personalized treatment, as well as timely adjustments to the treatment plan.

In some embodiments, the implantable neuromodulation module 1 is configured to obtain monitoring data generated during sacral nerve conduction via an implantable electrode and, simultaneously obtain a local field potential variation signal generated in a patient with urinary tract dysfunction during the sacral nerve conduction by monitoring. The local field potential variation signal is caused by a neural activity that leads to urinary tract dysfunction.

The implantable electrode refers to a microelectronic device that is surgically implanted into specific tissues or the nervous system within the body to monitor and record nerve signals or modulate physiological functions via electrical stimulation. The structure of the implantable electrode includes an electrode assembly, a signal processing circuit, a power supply, and a package structure. The system may modulate the nerve reflex pathways and improve the functional coordination of the bladder, urethra, and pelvic floor muscles through continuous or intermittent electrical stimulation to the sacral nerves (S3 or S4) via the implantable electrode. For example, for overactive bladder (OAB), the system may inhibit excessive contraction of the detrusor muscle and reduce urinary urgency and incontinence. As another example, for urinary retention, the system may enhance the contraction force of the detrusor muscle to promote urinary drainage.

The monitoring data generated during the sacral nerve conduction refers to data obtained by real-time recording and analysis of an electrical activity, a conduction feature, and a functional state of the sacral nerves (including sacral nerve roots, sacral nerve plexus, and their branches). The monitoring data may include a bioelectrical signal (e.g., nerve conduction velocity, electromyography, F-wave vs. H-wave reflex, membrane potential, etc.), neuroimaging data, and real-time feedback data in neuromodulation (e.g., frequencies, voltages, pulse widths, etc.). In some embodiments, the monitoring data includes a bioelectrical signal near the sacral nerve during a symptomatic phase (e.g., a urinary urgency symptom) and an asymptomatic phase. For example, the monitoring data includes the membrane potential of the peripheral nerve of the patient.

The local field potential variation signal refers to a signal that reflects a change in the local field potential (LFP). LFP is used to reflect the coordinated activity of a neuron group in the sacral nerves, plexuses, or ganglia. For example, the LFP is closely related to neural modulation of urination and urine storage function.

In some embodiments, the implantable neuromodulation module 1 establishes a direct electrical signal interaction with the sacral nerve system via the implantable electrode, to monitor and modulate the activity of the sacral nerve in real time, obtain the monitoring data generated during the sacral nerve conduction, and synchronously obtain the local field potential variation signal generated in the patient with urinary tract dysfunction during the sacral nerve conduction by monitoring.

In some embodiments, the implantable neuromodulation module 1 is further configured to apply an electrical stimulation pulse to the sacral nerve of the patient when an abnormal monitoring result of nerve afferent impulses associated with the urinary urgency symptom of the patient is obtained based on the local field potential variation signal. A control signal of the electrical stimulation pulse is generated by the programming module 2. More descriptions regarding the programming module 2 may be found in the related descriptions below and the related sections.

The abnormal monitoring result refers to a result that indicates a deviation from the normal range in frequency, amplitude, an application time point, synchronization, or conduction feature of a nerve signal related to the micturition reflex (such as action potential, LFP, nerve conduction velocity, etc.) in the patient with the urinary urgency symptom. In some embodiments, the abnormal monitoring result may include the abnormal bioelectrical signal, abnormal nerve conduction velocity and reflex, etc. For example, the abnormal monitoring result may manifest as an abnormal LFP spectrum, slowed nerve conduction velocity, or abnormal sacral nerve stimulation response, or the like.

The electrical stimulation pulse refers to a short, controlled current or voltage signal generated by a pulse generator (implantable or external). The electrical stimulation pulse is delivered to the sacral nerve via the implantable electrode to modulate the excitation or inhibition state of the neuron. The electrical stimulation pulse has a specific frequency, amplitude (voltage or current), pulse width (duration), and waveform, which is used to modulate abnormal neural reflex arcs and improve the function of target organs (such as the bladder, urethra, and pelvic floor muscles).

In some embodiments, the implantable neuromodulation module 1 may obtain the abnormal monitoring result of the nerve afferent impulses associated with the urinary urgency symptom of the patient by analyzing the features, such as the spectral, amplitude, phase, etc., of the local field potential. For example, if the power of the y frequency band in the local field potential increases by 50% or more compared to the baseline when the patient is in an episode of urinary urgency and a duration exceeds 5 seconds, the implantable neuromodulation module 1 may define the above monitoring result as an abnormal monitoring result.

In some embodiments, the abnormal monitoring result of the nerve afferent impulses associated with the urinary urgency symptom of the patient is generally caused by neural activity leading to abnormal lower urinary tract activity. The neural activity may be intervened by applying the electrical stimulation pulse to restore balance to the entire pelvic floor sacral nerve network and achieve a treatment effect. For example, after analyzing the sacral nerve data of a group of patients, it is found that when the patient feels the urge to urinate, the signal intensity of the local field potential variation signal after processing may show a slight increase in the 80-120 Hz frequency band. Based on this finding, a feasible implementation manner is to compare the baseline intensity with the real-time signal intensity. When the signal intensity increases, it is determined as an abnormal monitoring result of the nerve afferent impulses associated with the urinary urgency symptom of the patient, and the electrical stimulation function is activated accordingly. The foregoing embodiments are provided only for case of understanding and simplification of description and are not intended to be a limitation of the present disclosure.

In some embodiments, the implantable neuromodulation module 1 is further configured to deactivate, in response to recognizing the normal monitoring result, the electrical stimulation pulse applied to the sacral nerve after a preset delay time when the abnormal monitoring result returns to a normal monitoring result during the asymptomatic phase of the patient. In other words, the system automatically initiates the electrical stimulation pulse to the sacral nerve by the implantable neuromodulation module 1 when the abnormal monitoring result is recognized, and the system automatically deactivates the electrical stimulation pulse to the sacral nerve by the implantable neuromodulation module 1 when the abnormal monitoring result returns to the normal monitoring result. In some embodiments, a delay time may be set when deactivating the sacral nerve electrical stimulation pulse to achieve the delayed deactivating function of the electrical stimulation modulation, thereby ensuring the treatment effect of the electrical stimulation.

The normal monitoring result during the asymptomatic phase of the patient indicates that when the patient has no urinary urgency symptom, the nerve signal related to the micturition reflex (such as the action potential, the LFP, the nerve conduction velocity, etc.) is within a physiological range without an abnormal fluctuation or a pathological feature. For example, during the asymptomatic phase, the power in each band of the spectrum of the local field potential is at a baseline level, with no significant high-frequency or low-frequency abnormality, and the phase synchronization is moderate. As another example, the conduction velocity of the sacral nerve afferent fiber is within a normal range (e.g., the conduction velocity of the sacral nerve afferent fiber is larger than or equal to 40 m/s).

The preset delay time refers to a preconfigured interval during which the implantable neuromodulation module 1 postpones executing the deactivation of the sacral nerve electrical stimulation pulse after its trigger, thereby implementing the delay function. In the sacral nerve electrical stimulation, the preset delay time is used to control the deactivation timing of the electrical stimulation pulse. For example, the delay time may be set to 10 seconds.

In some embodiments, the implantable neuromodulation module 1 is further configured to determine whether the abnormal monitoring result of the nerve afferent impulses associated with the urinary urgency symptom of the patient exists based on a first processing result of the local field potential variation signal and an intelligent recognition model. When the implantable neuromodulation module 1 operates in conjunction with the programming module 2, the local field potential variation signal obtained from monitoring may be sent to the programming module 2.

More descriptions regarding the programming module 2 and the intelligent recognition model may be found in the related descriptions below and the related sections.

In some embodiments, the first processing result refers to a result after processing and/or feature extraction of the local field potential variation signal. For example, the first processing result may include a spectral feature, a time-frequency feature, a phase synchronization, a non-linear feature, or the like.

In some embodiments, the implantable neuromodulation module 1 may input the first processing result (e.g., a power spectral density, a phase synchronization matrix, etc.) of the local field potential variation signal into the intelligent recognition model, and the intelligent recognition model outputs an “abnormal” or “normal” classification result.

In some embodiments, the implantable neuromodulation module 1 may input the first processing result (e.g., a power spectral density, a phase synchronization matrix, etc.) of the local field potential variation signal into the intelligent recognition model, and the intelligent recognition model outputs an “abnormal” probability. If the “abnormal” probability is greater than or equal to a first preset threshold, the first processing result is classified as an abnormal monitoring result. The first preset threshold may be set manually or by the system. For example, the first preset threshold may be 0.8.

In some embodiments, the implantable neuromodulation module 1 determines whether the abnormal monitoring result of the nerve afferent impulses associated with the urinary urgency symptom of the patient exists based on the first processing result of the local field potential variation signal and the intelligent recognition model, and sends the local field potential variation signal obtained from monitoring to the programming module 2, thereby achieving real-time monitoring and early warning of the abnormal nerve afferent impulses associated with the urinary urgency symptom. At the same time, based on the analysis of the local field potential variation signal, the physician may understand the specific condition and etiology of the urinary urgency symptom of the patient to formulate a personalized treatment plan.

In some embodiments, the implantable neuromodulation module 1 is further configured to predict, by a prediction model, the application time points of the electrical stimulation pulse to the sacral nerve of patients with urinary tract dysfunction based on biological signals near the sacral nerve, bladder pressure data, patient information, and environmental data collected over a preset period, and apply the electrical stimulation pulse to the sacral nerve of the patient with urinary tract dysfunction based on the predicted application time points of the electrical stimulation pulse.

The patient information may include age, gender, weight, pathological data, etc. The pathological data may include blood glucose, inflammatory factors, neurotransmitter levels (e.g., 5-HT, dopamine), etc. The environmental data may include temperature, humidity, etc., of the environment where the patient with urinary tract dysfunction is located. More descriptions regarding the bioelectrical signals may be found elsewhere in the present disclosure (e.g., the monitoring data and related descriptions thereof). More descriptions regarding the intravesical pressure data may be found elsewhere in the present disclosure (e.g., the pressure monitoring module 4 and related descriptions thereof).

In some embodiments, the application time point of the electrical stimulation pulse is also associated with stimulation-feedback time of a plurality of cycles. For example, an input of the prediction model may also include the stimulation-feedback time of the plurality of cycles. A count of the cycles may be set manually or by the system. For example, the count of the cycles may be set to 1, 2, 3, etc. The count of the cycles may be the same as a preset count of cycles. More descriptions regarding the preset count of cycles and the stimulation-feedback time may be found elsewhere in the present disclosure (e.g., a parameter configuration unit 24 and related descriptions thereof).

In some embodiments, in response to the existence of an abnormal monitoring result of the nerve afferent impulses associated with the urinary urgency symptom of the patient, the implantable neuromodulation module 1 may apply the electrical stimulation pulse to the sacral nerve of the patient with urinary tract dysfunction based on the predicted application time points of the electrical stimulation pulse.

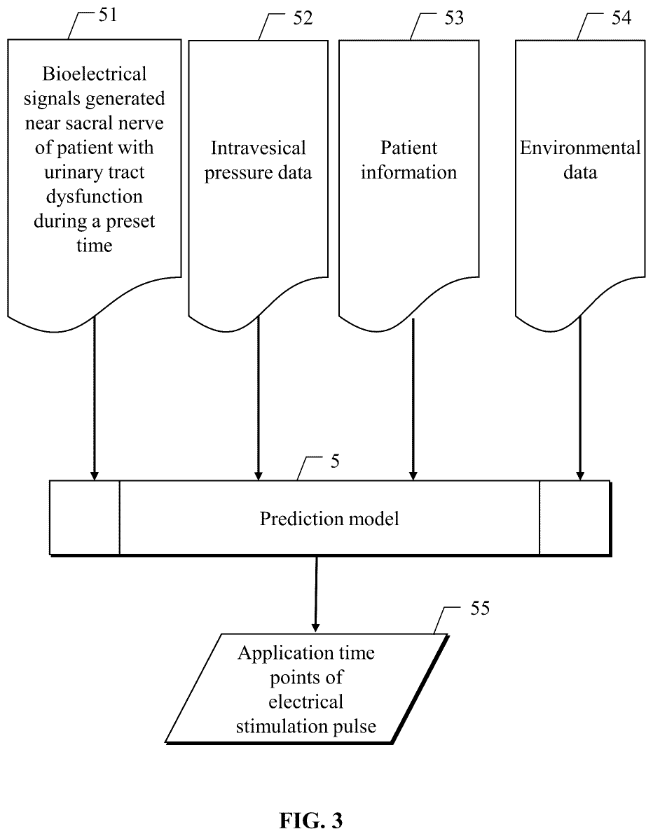

More descriptions regarding the prediction model may be found elsewhere in the present disclosure (e.g., FIG. 3 and related descriptions thereof).

The embodiments of the present disclosure further introduce the stimulation-feedback time of the plurality of cycles when predicting the application time points of the electrical stimulation pulse, which may reduce the duration of the abnormal state of the patient with urinary tract dysfunction.

In some embodiments, as shown in FIG. 1, the implantable neuromodulation module 1 includes an electrode channel 11, a sacral nerve signal acquisition unit 12, a first signal processing unit 13, a first feature extraction unit 14, an intelligent recognition unit 15, and a neurostimulation unit 16 connected in sequence.

The neurostimulation unit 16 is connected with the electrode channel 11 for applying the electrical stimulation pulse. The sacral nerve signal acquisition unit 12 is configured to acquire the local field potential variation signal generated during the sacral nerve conduction and transmit the local field potential variation signal to the first signal processing unit 13.

The first signal processing unit 13 performs filtering processing on the local field potential variation signal and sends the local field potential variation signal after processing to the first feature extraction unit 14. The filtering processing mainly removes noise and frequency signals unrelated to nerve conduction. The field potential around the nerve is generated by the collective activity of numerous nerve fibers, and it may manifest as a relatively high-frequency signal. Therefore, the filtering processing may better remove a fixed-frequency or lower-frequency signal related to respiration, heartbeat, and intestinal peristalsis, while retaining signals related to the nerve conduction. Signals related to nerve conduction refer to field potentials generated by the propagation of action potentials within axons. Afterwards, the first feature extraction unit 14 performs time-frequency processing on the signal after the filtering processing, extracts signal intensity features in unit time and segmented frequency bands to generate a two-dimensional feature matrix composed of time and frequency, extracts a first feature parameter, and sends the first feature parameter to the intelligent recognition unit 15. The first feature parameter refers to a feature parameter that may reflect a potential intensity variation and/or a potential frequency variation of the signal. By analyzing the first feature parameter with the intelligent recognition model, abnormal signals may be identified.

In some embodiments, the type of the first feature parameter may be set according to practical conditions, without limitation herein.

More descriptions regarding the electrode channel 11, the sacral nerve signal acquisition unit 12, the first signal processing unit 13, the first feature extraction unit 14, the intelligent recognition unit 15, and the neurostimulation unit 16 may be found in the related descriptions below and the related sections.

The electrode channel 11 refers to an independent pathway and/or unit that acquires, transmits, and records the bioelectrical signals or related physical and/or chemical signals via the implantable electrode. Each electrode channel 11 corresponds to an independent signal acquisition and processing line.

In some embodiments, the implantable neuromodulation module 1 further includes a sacral nerve signal acquisition unit 12. The sacral nerve signal acquisition unit 12 is configured to acquire, in real time, the local field potential variation signal generated by the patient with urinary tract dysfunction during the sacral nerve conduction, and send the local field potential variation signal to the first signal processing unit 13 of the implantable neuromodulation module 1 and the second signal processing unit 21 of the programming module 2, respectively. This enables real-time monitoring and dual-path parallel processing, thereby improving the timeliness of modulation. At the same time, the synergy of real-time processing and remote refinement analysis ensures the timeliness, safety, and personalization of modulation.

More descriptions regarding the second signal processing unit 21 of the programming module 2 may be found in the related descriptions below and the related sections.

In some embodiments, the implantable neuromodulation module 1 further includes the first signal processing unit 13. The first signal processing unit 13 is configured to perform first filtering processing on the local field potential variation signal and send the local field potential variation signal after processing to the first feature extraction unit 14. More descriptions regarding the first feature extraction unit 14 may be found in the related descriptions below and the related sections.

The first filtering processing refers to the processing of removing the noise and frequency signals unrelated to the nerve conduction in the local field potential variation signal and retaining the signals related to the nerve conduction. In some embodiments, the first signal processing unit 13 may perform signal segmentation and local characterization analysis on the local field potential variation signal to remove abnormal values, perform noise feature analysis through spectrum analysis (e.g., Fourier transform), and remove the noise through a filter (e.g., an adaptive filter).

In some embodiments, the implantable neuromodulation module 1 further includes the first feature extraction unit 14. The first feature extraction unit 14 is configured to perform the time-frequency processing on the local field potential variation signal after the first filtering processing to form the two-dimensional feature matrix of time and frequency, extract the first feature parameter, and send the first feature parameter to the intelligent recognition unit 15. More descriptions regarding the intelligent recognition unit 15 may be found in the related descriptions below and the related sections.

In some embodiments, the first feature extraction unit 14 segments the local field potential variation signal after the first filtering processing using a window function, performs a frequency domain analysis (e.g., the Fourier transform) on each segment of the signal, and then combines spectral results of each segment to form a joint time-frequency distribution. In some embodiments, the first feature extraction unit 14 may perform the time-frequency processing on the local field potential variation signal after the first filtering processing using continuous wavelet transform, synchro-squeezing transform, Hilbert transform, etc., to form the two-dimensional feature matrix of time and frequency.

The first feature parameter refers to a time-frequency correlation feature parameter. For example, the first feature parameter may include an energy peak feature, an energy change feature, a frequency time series, a time-frequency ratio feature, etc. In some embodiments, the first feature parameter may include at least one of the potential intensity variation or the potential frequency variation.

In some embodiments, the implantable neuromodulation module 1 includes the first feature extraction unit 14. The first feature extraction unit 14 is configured to perform the time-frequency processing on the local field potential variation signal after the first filtering processing to form the two-dimensional feature matrix of time and frequency, extract the first feature parameter, and send the first feature parameter to the intelligent recognition unit 15. In such a case, a dynamic change of the nerve signal may be captured, normal and abnormal neural activity may be accurately distinguished. Through pre-filtering and time-frequency feature extraction, the effective components and interference in the signal can be effectively separated, the robustness of neuromodulation can be enhanced, and the risk of misjudgment can be reduced. The two-dimensional feature matrix of time and frequency provides a multi-dimensional analysis dimension, which may be combined with the intelligent recognition model to identify a complex mode of the neural activity to support a finer tuning strategy. Meanwhile, converting a neuro-electrophysiological signal into a quantifiable and analyzable feature parameter can provide core support for the accuracy, real-time, intelligence, and safety of the closed-loop neuromodulation system based on sacral nerve electrophysiological activity monitoring.

In some embodiments, the intelligent recognition unit 15 is configured to identify, in real time, based on the intelligent recognition model, a first feature parameter corresponding to the local field potential variation signal generated by the patient with urinary tract dysfunction during the sacral nerve conduction. The first feature parameter includes at least one of the potential intensity variation or the potential frequency variation. More descriptions regarding the intelligent recognition model may be found elsewhere in the present disclosure (e.g., the server module 3 and related descriptions thereof).

In some embodiments, the intelligent recognition unit 15 is further configured to determine a first monitoring result corresponding to the local field potential variation signal based on the first feature parameter and initiate the electrical stimulation pulse to the sacral nerve when the abnormal monitoring result of the nerve afferent impulses associated with the urinary urgency symptom of the patient exists.

The first monitoring result of the local field potential variation signal refers to a result that may reflect a state of the neural activity, a specific physiological event, or a pathological change that is extracted by analyzing features (e.g., time, frequency, amplitude, phase, etc.) of the local field potential variation signal. In some embodiments, the first monitoring result may include the monitoring of neural rhythms and functional states, the monitoring of disease-related features, neuromodulation and feedback control, or the like. For example, the first monitoring result may include the abnormal monitoring result of the nerve afferent impulses associated with the urinary urgency symptom of the patient and/or the normal monitoring result during the asymptomatic phase of the patient.

In some embodiments, the intelligent recognition unit 15 may correlate the first feature parameter with a known neural activity or a clinical indicator, and determine, based on a second preset threshold or the intelligent recognition model, the first monitoring result corresponding to the local field potential variation signal. The second preset threshold may be set manually or by the system. For example, the intelligent recognition unit 15 may compare the potential intensity variation with a mean value of the potential intensity variation. If the potential intensity variation is greater than the mean value of the potential intensity variation, the abnormal monitoring result of the nerve afferent impulses associated with the urinary urgency symptom of the patient exists. As another example, the intelligent recognition unit 15 may input the first feature parameter into the intelligent recognition model, and the intelligent recognition model outputs a classification result of an “abnormal monitoring result” or “normal monitoring result”.

In some embodiments, the intelligent recognition unit 15 determines, in real time, whether the abnormal monitoring result of the nerve afferent impulses associated with the urinary urgency symptom of the patient exists in a current nerve signal based on the received first feature parameter and a constructed intelligent recognition model. For example, the intelligent recognition unit 15 inputs the first feature parameter into the constructed intelligent recognition model and then outputs a determination as to whether the abnormal monitoring result of the nerve afferent impulses associated with the urinary urgency symptom of the patient exists. When the abnormal monitoring result exists, the intelligent recognition unit 15 sends an instruction to the neurostimulation unit 16. The neurostimulation unit 16 applies the electrical stimulation pulse to the sacral nerves via the electrode channel 11 based on the electrical stimulation pulse parameter configured by the programming module 2, initiating the SNM.

More descriptions regarding the neurostimulation unit 16 and the programming module 2 may be found in the related descriptions below and the related sections.

The neurostimulation unit 16 refers to a unit that modulates, monitors, or treats neurological functions by applying a specific type of stimulation (e.g., the electrical stimulation pulse) to alter the membrane potential of a nerve cell, activate, or inhibit neural activity. For example, the neurostimulation unit 16 may be a neural stimulator, and the neural stimulator may include an electrical stimulator, an insulated puncture needle, an electrode lead, or the like.

Different from the problem of poor real-time performance of the acquisition and recognition process during the SNM in related technologies, the closed-loop neuromodulation system based on sacral nerve electrophysiological activity monitoring in the present disclosure obtains the monitoring data based on the implantable neuromodulation module 1, and obtains the local field potential variation signal generated in the patient with urinary tract dysfunction during the sacral nerve conduction by monitoring. The system applies the electrical stimulation pulse to the sacral nerve of the patient when the abnormal monitoring result of the nerve afferent impulses associated with the urinary urgency symptom of the patient is automatically identified, forming a complete closed-loop neuromodulation system, which effectively improves the treatment effectiveness.

The programming module 2 refers to a module that dynamically adjusts the sacral nerve electrical stimulation pulse parameter (e.g., a voltage, a pulse width, a frequency, etc.) through a programmed instruction. In some embodiments, the programming module 2 typically includes a microcontroller unit (MCU), a memory, a communication interface, a power management module, etc. The MCU serves as a core processing unit and is configured to adjust the electrical stimulation pulse parameter according to clinical needs. The memory is configured to save a treatment parameter and historical data of the patient, and the communication interface realizes wireless communication with an external device (e.g., a programmable controller, a display, etc.), which facilitates remote monitoring and parameter adjustment of the patient by healthcare personnel.

In some embodiments, the programming module 2 stimulates the sacral nerves by generating an electrical impulse of a specific frequency and intensity that affects a nerve conduction pathway, thereby modulating the functions of the relevant organs (e.g., bladder, urethral/anal sphincter, pelvic floor, etc.). For example, the programming module 2 receives an instruction from the programmable controller, adjusts the electrical stimulation pulse parameter, and transmits the signal to the implantable neuromodulation module 1 via wireless communication. The implantable neuromodulation module 1 then transmits the electrical impulse to the sacral nerve to achieve neuromodulation.

In some embodiments, the programming module 2 is configured to process the local field potential variation signal and determine whether an abnormal monitoring result of the nerve afferent impulses associated with the urinary urgency symptom of the patient exists based on a second processing result of the local field potential variation signal and the intelligent recognition model.

The second processing result is similar to the first processing result, and more descriptions regarding the first processing result may be found elsewhere in the present disclosure (e.g., the implantable neuromodulation module 1 and related descriptions thereof).

The process by which the programming module 2 determines whether the abnormal monitoring result of the nerve afferent impulses associated with the urinary urgency symptom of the patient exists is similar to the process by which the implantable neuromodulation module 1 determines whether the abnormal monitoring result exists, and more descriptions may be found elsewhere in the present disclosure (e.g., the implantable neuromodulation module 1 and related descriptions thereof).

In some embodiments, when the abnormal monitoring result of the nerve afferent impulses associated with the urinary urgency symptom of the patient exists, the programming module 2 is further configured to display corresponding interactive interface information and receive an electrical stimulation pulse parameter input from the physician to cause the implantable neuromodulation module to apply the electrical stimulation pulse to the sacral nerve.

The electrical stimulation pulse parameter refers to an electrical pulse signal feature sent via the implantable electrode and is configured to determine the intensity, frequency, and mode of the electrical stimulation pulse. The electrical stimulation pulse parameter may include a frequency, a pulse width, an amplitude, a stimulation cycle, an electrode configuration (e.g., a unipolar mode and/or a bipolar stimulation mode), etc. More descriptions regarding the electrical stimulation pulse parameter may be found in the related descriptions below and the related sections.

In some embodiments, as shown in FIG. 1, the programming module 2 is configured to receive the local field potential variation signal and perform signal processing while obtaining the constructed intelligent recognition model sent by the server module 3, determine whether the abnormal monitoring result of the nerve afferent impulses associated with the urinary urgency symptom of the patient exists based on the local field potential variation signal after processing and the constructed intelligent recognition model, and generate the electrical stimulation pulse parameter when the abnormal monitoring result of the nerve afferent impulses associated with the urinary urgency symptom of the patient exists.

In some embodiments, the programming module 2 includes a second signal processing unit 21, a second feature extraction unit 22, an algorithm configuration unit 25, a display unit 23, and a parameter configuration unit 24. The second signal processing unit 21 receives the local field potential variation signal and performs filtering processing on the local field potential variation signal, then sends the local field potential variation signal to the second feature extraction unit 22. The second feature extraction unit 22 performs the time-frequency processing on the signal after filtering processing, performs feature extraction on the signal intensity based on unit time (per frame) and segmented frequency bands (frequency division), forms the two-dimensional feature matrix of time and frequency, and extracts the second feature parameter.

The algorithm configuration unit 25 receives the second feature parameter extracted by the second feature extraction unit 22, obtains and stores the constructed intelligent recognition model sent by the server module 3, identifies the second feature parameter according to the constructed intelligent recognition model, and determines whether the abnormal monitoring result of the nerve afferent impulses associated with the patient's urinary urgency symptom. The display unit 23 is configured to display time-frequency information processed by the second feature extraction unit 22, and the time-frequency information includes, but is not limited to, a time-frequency diagram, or the like. When an abnormal monitoring result of the nerve afferent impulses associated with the urinary urgency symptom of the patient exists, the abnormal information is displayed by the display unit 23, and the abnormal information may be specifically marked in the time-frequency diagram for the physician to check.

In some embodiments, the first feature parameter is generally the same as the second feature parameter, and additionally, the implantable neuromodulation module 1 is similar to the programming module 2 in terms of the data processing operations, such as signal analysis and feature extraction, which are not repeated here.

More descriptions regarding the second signal processing unit 21, the second feature extraction unit 22, the algorithm configuration unit 25, the display unit 23, the parameter configuration unit 24, the server module 3, and the intelligent recognition model may be found in the related descriptions below and the related sections.

In some embodiments, when an abnormal monitoring result of the nerve afferent impulses associated with the urinary urgency symptom of the patient exists, the programming module 2 displays corresponding interactive interface information and receives the electrical stimulation pulse parameter input from the physician, to cause the implantable neuromodulation module to apply the electrical stimulation pulse to the sacral nerve. By monitoring abnormalities of the nerve afferent impulses, the programming module 2 may specifically adjust the electrical stimulation parameter (such as the frequency, the intensity, the pulse width, etc.) to act directly on the sacral nerve to modulate nerve reflexes of the bladder, the sphincter, and the pelvic floor. At the same time, the interactive interface allows the physician to dynamically adjust the electrical stimulation pulse parameter based on real-time monitoring data (e.g., abnormal patterns of nerve impulses) and symptom feedback of the patient, thereby enabling precise and personalized treatment.

In some embodiments, the programming module 2 further includes the second signal processing unit 21. The second signal processing unit 21 is configured to perform second filtering processing on the local field potential variation signal and send the local field potential variation signal after processing to the second feature extraction unit 22. More descriptions regarding the second feature extraction unit 22 may be found in the related descriptions below and the related sections.

The second filtering processing is similar to the first filtering processing, and more descriptions regarding the first filtering processing may be found elsewhere in the present disclosure (e.g., the first signal processing unit 13 and related descriptions thereof).

In some embodiments, the programming module 2 further includes the second feature extraction unit 22 and the algorithm configuration unit 25. The second feature extraction unit 22 is configured to perform the time-frequency processing on the local field potential variation signal after the second filtering processing, form the two-dimensional feature matrix of time and frequency, and extract the second feature parameter and send the second feature parameter to the algorithm configuration unit 25. More descriptions regarding the algorithm configuration unit 25 may be found in the related descriptions below and the related sections.

The process of the second feature extraction unit 22 performing the time-frequency processing on the local field potential variation signal after the second filtering processing is similar to the process of the first feature extraction unit 14 performing the time-frequency processing on the local field potential variation signal after the first filtering processing. More descriptions may be found elsewhere in the present disclosure (e.g., the first feature extraction unit 14 and related descriptions thereof).

The second feature parameter is similar to the first feature parameter, and more descriptions regarding the first feature parameter may be found elsewhere in the present disclosure (e.g., the first feature extraction unit 14 and related descriptions thereof).

In some embodiments, the programming module 2 further includes the display unit 23. The display unit 23 is configured to display the interactive interface information. The interactive interface information includes at least one of the local field potential variation signal transmitted by the implantable neuromodulation module 1, the second processing result and a second monitoring result of the local field potential variation signal obtained by the programming module 2, and a status interface of the implantable neuromodulation module 1.

In some embodiments, the interactive interface of the display unit 23 includes at least one of the local field potential variation signal transmitted by the implantable neuromodulation module 1, a computational processing result and a judgment result of the signal obtained by the second feature extraction unit 22 and the algorithm configuration unit 25, a selection interface of the intelligent recognition model of the algorithm configuration unit 25, an electrical stimulation parameter selection interface of the parameter configuration unit 24, the status interface of the implantable neuromodulation module 1, or the like. The display unit 23 is mainly configured to assist the other units in interacting with the physician. For example, the physician may select the intelligent recognition model and adjust a model parameter via the algorithm configuration unit 25, and input the personalized treatment parameters (e.g., the electrical stimulation pulse parameter), etc., for the patient via the parameter configuration unit 24.

In other words, the display unit 23 may, according to the needs of the user, display in real time the processing result of the signal acquisition, processing, and feature extraction, and a comprehensive judgment result of the intelligent recognition model, for professionals' reference to efficiently and accurately adjust the treatment parameter. The adjusted treatment parameter and intelligent recognition model may be sent to the implantable neuromodulation module 1 via the parameter configuration unit 24 and the algorithm configuration unit 25, respectively. Meanwhile, when the abnormal monitoring result of the nerve afferent impulses associated with the urinary urgency symptom of the patient exists, the physician may make a judgment based on the interactive interface information of the display unit 23, artificially configure the electrical stimulation pulse parameter via the parameter configuration unit 24, and send the electrical stimulation pulse parameter to the neurostimulation unit 16 of the implantable neuromodulation module 1, thereby causing the implantable neuromodulation module 1 to apply the corresponding electrical stimulation pulse according to the configured electrical stimulation pulse parameter.

More descriptions regarding the parameter configuration unit 24, the algorithm configuration unit 25, and the intelligent recognition model may be found in the related descriptions below and the related sections.

The second monitoring result is similar to the first monitoring result, and more descriptions regarding the second monitoring result may be found elsewhere in the present disclosure (e.g., the intelligent recognition unit 15 and related descriptions thereof).

The status interface of the implantable neuromodulation module 1 refers to an interface for display and interaction, which presents a real-time state, a setting of the parameter, etc., of the implanted neuromodulation components (e.g., the electrode channel 11, the neurostimulation unit 16, etc.) visually. In some embodiments, the status interface of the implantable neuromodulation module 1 may include a parameter adjustment area, a status feedback area, an alert area, or the like.

The general modulation manner used in the related art has problems of lacking individualized precision or having poor individual precision when performing SNM and causing patient discomfort and power consumption by continuous electrical stimulation. Different from the general modulation manner, the closed-loop neuromodulation system based on sacral nerve electrophysiological activity monitoring in the present disclosure displays the local field potential variation signal and its monitoring results in real time via the display unit 23 in the programming module, which can better help researchers or physicians to study and determine the correlation between the specificity of the local field potential change of the sacral nerve and the symptomatic phase of the patient. More importantly, the present disclosure can realize on-demand stimulation, intermittent stimulation, and quantitative precision stimulation based on individual bioelectrical signal indicators, avoid ineffective stimulation, reduce stimulation discomfort of the patient, and extend the battery life of the electrical control system.

In some embodiments, the programming module 2 further includes the parameter configuration unit 24. The parameter configuration unit 24 is configured to configure the electrical stimulation pulse parameter based on the interactive interface information of the display unit 23 and send the electrical stimulation pulse parameter to the neurostimulation unit 16 of the implantable neuromodulation module 1, to apply the electrical stimulation pulse to the sacral nerve via the electrode channel 11.

In some embodiments, the electrical stimulation pulse parameter may be a standardized treatment parameter pre-stored in the parameter configuration unit 24, a treatment parameter automatically generated based on the monitoring data by a pre-established machine learning model, or the electrical stimulation pulse parameter comprehensively determined and set by the physician based on the interactive interface information of the display unit 23, which are not limited herein.

In some embodiments, the electrical stimulation pulse parameter contained in the parameter configuration unit 24 includes at least one of the amplitude, the pulse width, the frequency, a treatment duration, a variable/constant frequency, a high frequency (e.g., 15 Hz-200 Hz), a low frequency (e.g., 1-15 Hz), or a voltage/current source. In some embodiments, when the implantable neuromodulation module 1 is used offline, the sacral nerve electrical stimulation pulse parameter may be preset. For example, stimulation intensity is determined based on the patient's typical motor responses, sensory response, and a pre-wearing effect during a trial period. For example, the frequency is generally set to 14 Hz, the pulse width is set to 210 μs, which is not limited herein.

In some embodiments, the programming module 2 further includes the parameter configuration unit 24. The parameter configuration unit 24 is configured to configure the electrical stimulation pulse parameter (e.g., the amplitude, the pulse width, the frequency, the treatment duration, the variable/constant frequency, the high frequency, the low frequency, and the voltage/current source) based on the interactive interface information of the display unit 23, and send the electrical stimulation pulse parameter to the neurostimulation unit 16 of the implantable neuromodulation module 1, to apply the electrical stimulation pulse to the sacral nerve via the electrode channel 11, which can effectively intervene in the nerve impulse abnormality associated with the urinary urgency by precisely configuring the electrical stimulation pulse parameter. Meanwhile, the parameter configuration unit 24 can minimize discomfort while effectively relieving the symptom by configuring the electrical stimulation pulse parameter based on the real-time symptom feedback (e.g., a frequency variation of the urinary urgency), the monitoring data (e.g., intensity of the sacral nerve afferent impulses), etc., of the patient.

In some embodiments, the parameter configuration unit 24 is further configured to update the electrical stimulation pulse parameter based on a preset cycle T. The preset cycle T may be set manually or by the system. For example, the preset cycle T may be set to 1 hour, 3 hours, etc.

In some embodiments, during the i-th cycle Tᵢ (i is an integer greater than or equal to 0), the parameter configuration unit 24 is configured to update the electrical stimulation pulse parameter of the current cycle Tᵢ based on the abnormal monitoring result, the first feature parameter, and the second feature parameter of the previous cycle Tᵢ₋₁.

In some embodiments, the parameter configuration unit 24 may calculate a statistical value (e.g., a mean value, a median value, a variance, etc.) of an abnormal duration of the previous cycle Tᵢ₋₁ based on the abnormal monitoring result, the first feature parameter, and the second feature parameter of the previous cycle Tᵢ₋₁. If the statistical value of the abnormal duration is greater than the second preset threshold, the parameter configuration unit 24 may increase the amplitude, frequency, and treatment duration, etc., of the electrical stimulation pulse parameter in the previous cycle Tᵢ₋₁ based on an adjustment amplitude (e.g., +8%, −1%, etc.), to complete the updating of the electrical stimulation pulse parameter of the current cycle Tᵢ. The second preset threshold may be set manually or by the system. For example, the second preset threshold may be set to 10 seconds, 30 seconds, etc.

In some embodiments, the adjustment amplitude may be a preset value or a statistical value positively correlated to the abnormal duration. That is, the larger the statistical value of the abnormal duration, the larger the adjustment amplitude. The adjustment amplitude may also be adjusted manually. For example, the physician may adjust the stimulation intensity manually when it is excessively high or excessively low.

In some embodiments, the parameter configuration unit 24 is further configured to determine, based on the abnormal monitoring result, the first feature parameter, and the second feature parameter of the previous cycle Tᵢ₋₁, a stimulation-feedback time of the current cycle Tᵢ, and update the electrical stimulation pulse parameter of the current cycle Tᵢ based on the stimulation-feedback time of the current cycle Tᵢ.

The stimulation-feedback time refers to the response time of the patient with urinary tract dysfunction transforming from an abnormal state to a normal state after the implantable neuromodulation module 1 applies the electrical stimulation pulse.

In some embodiments, the parameter configuration unit 24 may determine, based on the abnormal monitoring result, the first feature parameter, and the second feature parameter of the previous cycle Tᵢ₋₁, the stimulation-feedback time of the current cycle Tᵢ by querying a first preset table. The first preset table includes a correspondence relation between the abnormal monitoring result, the first feature parameter, the second feature parameter, and the stimulation-feedback time. For example, the correspondence relation may be {index 1 (abnormal monitoring result), index 2 (first feature parameter), and index 3 (second feature parameter)->query result (stimulation-feedback time)}. In some embodiments, the first preset table may be constructed based on the historical data.

In some embodiments, the adjustment amplitude of the electrical stimulation pulse parameter is positively correlated with the degree to which the stimulation-feedback time exceeds a preset time threshold (e.g., the difference between the stimulation-feedback time and the preset time threshold). The preset time threshold may be set manually or by the system. For example, the preset time threshold may be set to 10 seconds, 30 seconds, etc.

In some embodiments, the larger the difference between the stimulation-feedback time and the preset time threshold, i.e., the more the stimulation-feedback time exceeds the preset time threshold, the larger the adjustment amplitude of the electrical stimulation pulse parameter. The parameter configuration unit 24 may increase the amplitude, the frequency, and the treatment duration, etc., of the electrical stimulation pulse parameter of the previous cycle Tᵢ₋₁ based on the adjustment amplitude of the electrical stimulation pulse parameter, to complete the updating of the electrical stimulation pulse parameter of the current cycle Tᵢ.

In some embodiments, the parameter configuration unit 24 is further configured to determine a stimulus adaptation of the current cycle Tᵢ based on the first feature parameter and the second feature parameter of a preset count of cycles (e.g., i cycles T₀˜Tᵢ₋₁), determine a stimulation mode of the current cycle Tᵢ based on the stimulus adaptation of the current cycle Tᵢ, and update the electrical stimulation pulse parameter of the current cycle Tᵢ based on the stimulation mode of the current cycle Tᵢ and the stimulation-feedback time of the current cycle Tᵢ.

The stimulus adaptation is used to quantify the resistance of the patient with urinary tract dysfunction to the electrical stimulation pulse. For example, the stimulus adaptation may be expressed as a value between 0 and 1. The closer the stimulus adaptation is to 1, the greater the resistance of the patient with urinary tract dysfunction to the electrical stimulation pulse. The preset count of cycles may be set manually or by the system.

In some embodiments, the parameter configuration unit 24 is further configured to calculate the amplitude change of the first feature parameter and/or the second feature parameter of two adjacent cycles in the preset count of cycles. The greater the average amplitude change of the first feature parameter and/or the second feature parameter of the two adjacent cycles, the greater the stimulus adaptation.

For example, the preset count of cycles is 4, i.e., 4 preset cycles T₁, T₂, T₃ and T₄ exist, and the first feature parameters corresponding to the 4 preset cycles T₁, T₂, T₃ and T₄ are a₁, a₂, a₃ and a₄, respectively, then the amplitude change of the first feature parameter of two adjacent cycles are (a₂−a₁), (a₃−a₂), and (a₄−a₃), respectively, and the average amplitude change is [(a₂−a₁)+(a₃−a₂)+(a₄−a₃)]/3.

In some embodiments, the parameter configuration unit 24 is further configured to calculate a symptom fluctuation value of the patient with urinary tract dysfunction and determine the preset count of cycles (i.e., a preset number of cycles) based on the symptom fluctuation value.

In some embodiments, the preset count of cycles is negatively correlated to the symptom fluctuation value. For example, the greater the symptom fluctuation value, the smaller the preset count of cycles.

In some embodiments, the parameter configuration unit 24 may obtain, on a daily basis, a count of the abnormal monitoring result that occurs in the patient with urinary tract dysfunction in recent days. The count of the abnormal monitoring result may be denoted as n1, n2, etc. The parameter configuration unit 24 may calculate a variance and/or standard deviation of the count of the abnormal monitoring result n1, n2, etc., and designate the variance and/or standard deviation as the symptom fluctuation value.

The stimulation mode may include a stimulation holiday mode and an augmented stimulation mode. The stimulation holiday mode refers to a cessation of the electrical stimulation pulse. The augmented stimulation mode refers to the increase in the intensity of the electrical stimulation pulse.

In some embodiments, the parameter configuration unit 24 is further configured to switch the stimulation mode to the stimulation holiday mode in response to determining that the stimulus adaptation is greater than a preset stimulation threshold and convert the stimulation mode to the augmented stimulation mode in response to determining that the stimulus adaptation is less than or equal to the preset stimulation threshold. The preset stimulation threshold may be set manually or by the system. For example, the preset stimulation threshold may be set to 0.5, 0.8, etc.

In some embodiments, the parameter configuration unit 24 may update, based on the stimulation mode of the current cycle Tᵢ and the stimulation-feedback time of the current cycle Tᵢ, the electrical stimulation pulse parameter of the current cycle Tᵢ by querying a second preset table. The second preset table includes a correspondence relation between the stimulation mode of the current cycle Tᵢ, the stimulation-feedback time of the current cycle Tᵢ, and the electrical stimulation pulse parameter of the current cycle Tᵢ. For example, the correspondence relation may be {index 1 (stimulation mode of the current cycle Tᵢ) and index 2 (stimulation-feedback time of the current cycle Tᵢ)->query result (electrical stimulation pulse parameter of the current cycle Tᵢ)}. In some embodiments, the second preset table may be constructed based on the historical data.

In some embodiments, the parameter configuration unit 24 may periodically adjust and update the electrical stimulation pulse parameter based on the preset cycle T, thereby adapting the electrical stimulation pulse parameter to a real-time change and avoiding inflexibility caused by the fixed electrical stimulation pulse parameter, ensuring that the adjustment of the electrical stimulation pulse parameter is more humanized and personalized.

The algorithm configuration unit 25 refers to a module or unit configured to manage and store information such as algorithm parameters, running configurations, input/output paths, logic rules, or the like. In some embodiments, the algorithm configuration unit 25 may be configured for parameterization management (e.g., the second feature parameter), environment configuration (e.g., a data path, hardware configuration, etc.), logic switching, version control, etc.

In some embodiments, the programming module 2 further includes the algorithm configuration unit 25. The algorithm configuration unit 25 is configured to identify, in real time, based on the intelligent recognition model, the second feature parameter corresponding to the local field potential variation signal generated by the patient with urinary tract dysfunction during the sacral nerve conduction. The second feature parameter includes at least one of the potential intensity variation or the potential frequency variation.

In some embodiments, the algorithm configuration unit 25 is further configured to determine the second monitoring result corresponding to the local field potential variation signal based on the second feature parameter, adjust the model parameter of the intelligent recognition model, and send the adjusted intelligent recognition model to the implantable neuromodulation module 1.

The process by which the algorithm configuration unit 25 determines the second monitoring result corresponding to the local field potential variation signal based on the second feature parameter is similar to the process by which the intelligent recognition unit 15 determines the first monitoring result, and more descriptions may be found elsewhere in the present disclosure (e.g., the intelligent recognition unit 15 and related descriptions thereof).

The model parameter of the intelligent recognition model may include a structural parameter and a hyperparameter of the intelligent recognition model. The hyperparameter is used to control a model training process and a learning strategy. In some embodiments, the intelligent recognition model is a machine learning model, e.g., selective logistic regression, a support vector machine (SVM), a random forest, neural network (e.g., a multilayer perceptron (MLP), a convolutional neural network (CNN)), etc. The structural parameter of the intelligent recognition model may include a count of layers, a count of neurons per layer, a count of filters in a convolutional layer, a type of pooling layer (e.g., max pooling, average pooling, etc.), a size of pooling window, or the like. The hyperparameter of the intelligent recognition model may include a count of training iterations, a loss function parameter, a decision threshold, or the like.

In some embodiments, the algorithm configuration unit 25 may adjust the model parameter of the intelligent recognition model based on the potential intensity variation and/or the potential frequency variation. For example, when a sudden change (e.g., a sudden rise) exists in the potential intensity or frequency, the algorithm configuration unit 25 may increase the learning rate of the intelligent recognition model to quickly capture the change and increase the count of training iterations to refine the gradient update. As another example, when a count of the high frequency signals increases, the algorithm configuration unit 25 may increase the weight or kernel size of the high frequency filter. As yet another example, the algorithm configuration unit 25 may dynamically adjust a classification threshold based on a potential intensity distribution. Merely by way of example, the threshold is increased in a high intensity range to reduce false positives, and the threshold is decreased in a low intensity range to capture a weak signal.

In some embodiments, the algorithm configuration unit 25 identifies, in real time, based on the intelligent recognition model, the second feature parameter corresponding to the local field potential variation signal generated by the patient with urinary tract dysfunction during the sacral nerve conduction, determines the second monitoring result corresponding to the local field potential variation signal based on the second feature parameter, and adjusts the model parameter of the intelligent recognition model. A subtle change during the sacral nerve conduction may be captured, then the abnormal neural activity is accurately located. At the same time, the closed-loop neuromodulation system based on sacral nerve electrophysiological activity monitoring can optimize the stimulation settings automatically based on the updated model parameter, thus reducing the diagnosis and treatment time and labor cost.

In some embodiments, the implantable neuromodulation module 1 may compare the first processing result with the first feature parameter identified by the intelligent recognition model to obtain a first difference value. The programming module 2 may compare the second processing result with the second feature parameter identified by the intelligent recognition model to obtain a second difference value. In response to determining that both the first difference and second difference are not greater than a preset threshold K, the system (i.e., the closed-loop neuromodulation system based on sacral nerve electrophysiological activity monitoring) adopts the abnormal monitoring result fed back by the implantable neuromodulation module 1. In response to determining that both the first difference and the second difference are greater than the preset threshold K, the system does not provide feedback on the abnormal monitoring result. In response to determining that the first difference is not greater than the preset threshold K and the second difference is greater than the preset threshold K, the system adopts the abnormal monitoring result fed back by the implantable neuromodulation module 1. In response to the first difference being greater than the preset threshold K and the second difference not being greater than the preset threshold K, the system adopts the abnormal monitoring result fed back by the programming module 2. The preset threshold K may be set based on experience or by the system. For example, the preset threshold K may be set to 80 microvolts.

In some embodiments, the closed-loop neuromodulation system based on sacral nerve electrophysiological activity monitoring further includes the server module 3. The server module 3 may include a processor unit, a memory unit, a storage unit, a network interface, or the like. In some embodiments, the server module 3 is configured to store the monitoring data and send an intelligent recognition model that is pre-trained to the implantable neuromodulation module 1 and the programming module 2, respectively.

In some embodiments, the implantable neuromodulation module 1 acquires and processes the local field potential variation signal generated during the sacral nerve conduction and sends the local field potential variation signal to the programming module 2, and the programming module 2 continues to send the local field potential variation signal to the server module 3. In other words, the embodiments of the present disclosure monitor the bioelectrical signals generated near the sacral nerve of the patient during the symptomatic phase and the asymptomatic phase by implantation (e.g., monitoring the membrane potential of the peripheral nerve of the patient). After that, the server module 3 is configured to receive the local field potential variation signal sent by the programming module 2 to train a classifier to construct the intelligent recognition model, and send the constructed intelligent recognition model to the programming module 2. Meanwhile, the programming module 2 also sends the constructed intelligent recognition model to the implantable neuromodulation module 1, for example, sends the constructed intelligent recognition model to the intelligent recognition unit 15 after sending the constructed intelligent recognition model to the algorithm configuration unit 25. More descriptions regarding the construction of the intelligent recognition model may be found in the related descriptions below and the related sections.

In some embodiments, the implantable neuromodulation module 1 obtains the constructed intelligent recognition model sent by the programming module 2, to automatically determine whether an abnormal monitoring result of the nerve afferent impulses associated with the urinary urgency symptom of the patient exists based on the signal processing result and the constructed intelligent recognition model. The implantable neuromodulation module 1 sends the local field potential variation signal to the programming module 2 and obtains the electrical stimulation pulse parameter generated by the programming module 2, i.e., the control signal of the electrical stimulation pulse is sent by the programming module 2. When an abnormal monitoring result of the nerve afferent impulses associated with the urinary urgency symptom of the patient exists, the electrical stimulation pulse function is initiated, and the electrical stimulation pulse is applied to the sacral nerve according to the electrical stimulation pulse parameter.

In some embodiments, the server module 3 is further configured to construct the intelligent recognition model. The intelligent recognition model is obtained by training using a plurality of sets of training data through a machine learning algorithm. Each set of training data among the plurality of sets of training data includes the bioelectrical signals generated near the sacral nerve of the patient with urinary tract dysfunction during the symptomatic phase and the asymptomatic phase.

In some embodiments, the intelligent recognition model is constructed based on the classifier. In the model training phase, the classifier is trained based on the machine learning model using the monitoring data stored on the server module 3 as a training set. The monitoring data includes the bioelectrical signals near the sacral nerves during the symptomatic phase and the asymptomatic phase.

The training process also includes denoising, band-pass filtering processing, and amplification, and only the signal related to the nerve conduction is retained. Then, the signal is subjected to time-frequency analysis, and the signal intensity is extracted in time units and different frequency bands to obtain a two-dimensional feature parameter

$$V = [ \begin{matrix}{v}_{11} & … & {v}_{1 j} \\ ⋮ & ⋱ & ⋮ \\ {v}_{i 1} & … & {v}_{ij}\end{matrix} ]$$

of the entire signal. i denotes the dimension of time resolution in the time domain, and j denotes the dimension of frequency resolution in the frequency domain. The set V is divided into a plurality of small sets with equal rows and columns, i.e.,

$$V = [ \begin{matrix}{V}_{11} & … & {V}_{1 l} \\ ⋮ & ⋱ & ⋮ \\ {V}_{k 1} & … & {V}_{kl}\end{matrix} ] , and {V}_{11} = [ \begin{matrix}{v}_{11} & … & {v}_{1 n} \\ ⋮ & ⋱ & ⋮ \\ {v}_{m 1} & … & {v}_{mn}\end{matrix} ] .$$

The plurality of small sets form a training sample set, which is used to train the classifiers, such as a convolutional neural network or a graph neural network.

In some embodiments, a dependent variable for training the convolutional neural network may be a signal of the patient during an episode of urinary urgency, and the purpose is to use the model to predict the signal change during the episode of urinary urgency. The two-dimensional feature matrix obtained after the time-frequency analysis may reflect a feature of the nerve signal in a current time window, and the two-dimensional feature matrix is a time-frequency diagram. Since the convolutional neural network or the graph neural network has a good recognition effect in image feature extraction, such a design is more conducive to identifying an effective nerve signal.

In some embodiments, taking the dimension 2048*2048 (i.e., i=j=2048) of a typical graph processing as an example, the dimension of a subset is set to 256*256, and when there is no overlapping of subset data, k=i/m=8 and 1=j/n=8. i, j, m, and n are all integers. Therefore, by training the configured classifier based on a received local field potential variation signal and sending the constructed intelligent recognition model after the iteration to the programming module 2, it is possible to determine, in real time, whether an abnormality in the current nerve signal exists, which not only improves the accuracy of the module training, but also realizes personalized treatment for the patient. For example, in the training of the classifier, an input of the classifier is monitoring data, and an output of the classifier is a recognition result of the abnormal monitoring result with a classification label.

The above descriptions of the training process of the intelligent recognition model, the types of the classifier, or the like, are merely exemplary and cannot be construed as a limitation of the present disclosure.

Different from the manual recognition or self-supervision model in the related technology, which has a poor recognition effect, the closed-loop neuromodulation system based on sacral nerve electrophysiological activity monitoring in the present disclosure stores the monitoring data via the server module 3 and sends the intelligent recognition model that is pre-trained to the implantable neuromodulation module 1 and the programming module, respectively. The closed-loop neuromodulation system may determine, in real time, whether an abnormality exists in the current nerve signal, thereby improving the efficiency and accuracy of recognition.

From the above, it may be seen that the modulation process of the closed-loop neuromodulation system based on sacral nerve electrophysiological activity monitoring of the embodiment of the present disclosure is as follows.

Firstly, the implantable neuromodulation module 1 acquires and processes the local field potential variation signal generated during the sacral nerve conduction and sends the local field potential variation signal to the programming module 2, and the programming module 2 continues to send the local field potential variation signal to the server module 3. Secondly, the server module 3 performs classifier training on the received signal and sends the constructed intelligent recognition model to the programming module 2 and the implantable neuromodulation module 1, respectively. After that, the implantable neuromodulation module 1 determines whether an abnormal monitoring result of the nerve afferent impulses associated with the urinary urgency symptom of the patient exists based on the processing result of the local field potential variation signal and the constructed intelligent recognition model. When the abnormal monitoring result of the nerve afferent impulses associated with the urinary urgency symptom of the patient exists, the electrical stimulation intervention is initiated. At the same time, the programming module 2 also determines whether the abnormal monitoring result of the nerve afferent impulses associated with the urinary urgency symptom of the patient exists based on the processing result of the local field potential variation signal and the constructed intelligent recognition model. When the abnormal monitoring result of the nerve afferent impulses associated with the urinary urgency symptom of the patient exists, the display unit 23 of the programming module 2 displays the interactive interface information. Finally, the electrical stimulation pulse parameter is comprehensively judged and set according to the pre-stored standardized treatment parameter, or an automatically generated electrical stimulation pulse parameter, or from the physician's input based on the interactive interface information of the display unit 23. The above electrical stimulation pulse parameter is received by the implantable neuromodulation module 1, and the electrical stimulation intervention is initiated according to the electrical stimulation pulse parameter.

In some embodiments, the closed-loop neuromodulation system based on sacral nerve electrophysiological activity monitoring further includes the pressure monitoring module 4. The pressure monitoring module 4 is configured to monitor the intravesical pressure data in real time and determine the preset delay time based on the intravesical pressure data within a preset time.

In some embodiments, the pressure monitoring module 4 may be a wearable bladder pressure monitoring device and a wireless bladder pressure monitoring device. In some embodiments, the pressure monitoring module 4 may be an independent module or a unit of the implantable neuromodulation module 1.

In some embodiments, if the pressure monitoring module 4 is an independent module, the pressure monitoring module 4 is configured to send the intravesical pressure data to the server module 3. The server module 3 sends the intravesical pressure data to the implantable neuromodulation module 1, thereby determining the preset delay time based on the intravesical pressure data within the preset time.

The preset time refers to a period with a preset duration. For example, the preset time may be a preset historical period. The intravesical pressure data refers to a pressure value measured inside the bladder, which is used to evaluate the urine storage and urination functions of the bladder. For example, the intravesical pressure data may include an amplitude change of the intravesical pressure, a variance of the intravesical pressure, or the like. In some embodiments, the preset delay time is positively correlated with the intravesical pressure data. For example, the smaller the amplitude change or the variance of the intravesical pressure, the shorter the preset delay time.

In other words, both the implantable neuromodulation module 1 and the programming module 2 may process raw signal data and obtain a processing result and a judgment result, respectively. Such a design not only ensures the offline use of the implantable neuromodulation module 1 but also improves computing power and operation speed of the system. For example, the implantable neuromodulation module 1 may only process real-time data of the patient each time or at one monitoring stage, and the programming module 2 displays aggregated raw signal data, the processing result, and the judgment result, etc., to the physician for reference and sends the above data to the server module 3 to store and update the model parameter.

As illustrated, the closed-loop neuromodulation system based on sacral nerve electrophysiological activity monitoring in the embodiments of the present disclosure may acquire in real-time, monitor, and automatically identify the local field potential variation signal generated in the patient during the sacral nerve conduction, and automatically apply the electrical stimulation for intervention when the abnormal electrical signal exists, thereby forming a complete closed-loop neuromodulation. Thus, the accuracy of the system recognition is effectively improved, on-demand acquisition and quantitative and precise stimulation based on the individual bioelectrical signal indicators are realized, further enhancing the treatment ability and treatment effect. At the same time, since the implantable neuromodulation module 1 needs to be implanted in the body of the patient, the on-demand stimulation, intermittent stimulation, and quantitative and precise stimulation functions of the present disclosure can effectively minimize patient discomfort caused by electrical stimulation while extending the battery life of the neuromodulation system. The need for frequent replacement of the implanted device is not required, thereby improving the treatment experience of the patient.

It should be noted that the present disclosure is particularly suitable for the monitoring, symptom recognition, assessment, and electrical stimulation therapy of the patient with OAB. Certainly, the technical solution of the present disclosure may also be extended to the monitoring and treatment of other urinary tract dysfunction symptoms, which are not limited here.

It should be noted that the above description of the closed-loop neuromodulation system based on sacral nerve electrophysiological activity monitoring and the modules of the closed-loop neuromodulation system are only for the convenience of description and cannot limit the present disclosure to the scope of the embodiments cited. It is to be understood that for those skilled in the art, after understanding the principle of the system, it may be possible to arbitrarily combine individual modules or form a subsystem to connect with other modules without departing from this principle. In some embodiments, the implantable neuromodulation module 1, the programming module 2, and the server module 3 disclosed in FIG. 1 may be different modules in a single system or a single module realizing the functions of two or more of the above modules. For example, each module may share a common storage module, and each module may also have a respective storage module. Such morphisms are within the scope of protection of the present disclosure.

FIG. 2 is a diagram illustrating a potential signal change during a urinary urgency phase and a non-urinary urgency phase of a patient after electrode implantation according to some embodiments of the present disclosure.

Referring to FIG. 2, the left panel of FIG. 2 is an original potential signal, with a horizontal axis indicating time, and a vertical axis indicating voltage (with a unit of mV). The right panel of FIG. 2 is a spectrum analysis diagram corresponding to the original potential signal, with a horizontal axis indicating frequency (with a unit of Hz), and a vertical axis indicating signal intensity (with a unit of dB).

As shown in FIG. 2, after monitoring local field potential signals near the sacral nerve root during both the urinary urgency phase and non-urinary urgency phase, original potential signals are subjected to the spectrum conversion. The intensity of the low-frequency signal of the sacral nerve root signal in patients in the urinary urgency phase is decreased, and the intensity of the high-frequency signal is increased. Therefore, in the state of urinary urgency, the local field potential of the sacral nerve root in patients shows an increase in input and output of the high-frequency signal and a decrease in input and output of the low-frequency signal.

FIG. 3 is a schematic diagram illustrating an exemplary prediction model according to some embodiments of the present disclosure.

As shown in FIG. 3, in some embodiments, a prediction model 5 may be a machine learning model, e.g., the prediction model 5 may include any one or a combination of a neural network (NN) model, a deep neural network (DN) model, or other customized models, etc. An input of the prediction model 5 may include bioelectrical signals 51 generated near the sacral nerve of the patient with urinary tract dysfunction during a preset time, intravesical pressure data 52, patient information 53, and environmental data 54. An output of the prediction model 5 may include application time points 55 of the electrical stimulation pulse.

The prediction model 5 may be obtained by training based on at least one group of training samples with a label. In some embodiments, the training sample may include at least one group of sample historical bioelectrical signals, sample historical intravesical pressure data, sample historical patient data, and sample historical environmental data during a historical first period. The label of the training samples may be the application time points of the electrical stimulation pulse during a historical second period. The label of the training sample may determine, based on a first feature parameter and/or a second feature parameter (e.g., a potential intensity variation, a potential frequency variation) during the historical second period, whether an abnormal monitoring result of nerve afferent impulses associated with urinary urgency symptom of the patient exists by an intelligent recognition model, and designate a time point when the abnormal monitoring result first occurs as an application time point of the electrical stimulation pulse. The historical first period is before the historical second period. The label of the training sample is the application time points of the electrical stimulation pulse obtained based on the sample historical bioelectrical signals, the sample historical intravesical pressure data, the sample historical patient data, and the sample historical environmental data of the training samples, which can quickly and accurately adjust the electrical stimulation pulse applied to the sacral nerve of the patient.

When training the prediction model, the training samples are input into an initial prediction model, and a loss function is constructed based on an output of the initial prediction model and the label. Parameters of the initial prediction model are iteratively updated based on the loss function until the parameters of the initial prediction model satisfy a preset training condition, then the training is completed, a trained prediction model is obtained, and the trained prediction model is designated as the prediction model 5. The preset training condition may include, but is not limited to, the loss function converging, the training cycle reaching a threshold, or the like. In some embodiments, similar to the intelligent recognition model, the server module 3 is further configured to construct the prediction model 5 (i.e., the training of the prediction model 5 may be completed by the server module 3) and send the pre-trained prediction model 5 to the implantable neuromodulation module 1 and the programming module 2, respectively.

The embodiment of the present disclosure utilizes the prediction model 5 to predict the application time points of the electrical stimulation pulse. The prediction based on the bioelectrical signals 51 generated near the sacral nerve, the intravesical pressure data 52, the patient information 53, and the environmental data 54 from the patient with urinary dysfunction over a preset period of time, thereby predicting possible application time points of the electrical stimulation pulse in advance. When the abnormal monitoring result of the nerve afferent impulses associated with the urinary urgency symptom of the patient is detected, the embodiment of the present disclosure may apply the electrical stimulation pulse at the predicted application time points of the electrical stimulation pulse based on the previously determined electrical stimulation pulse parameter, thereby enabling fast, accurate, and automatic adjustment of the electrical stimulation pulse.

In summary, the present disclosure achieves at least the following technical effects.

First, the closed-loop neuromodulation system based on sacral nerve electrophysiological activity monitoring in the embodiments of the present disclosure acquires the local field potential variation signal generated during the sacral nerve conduction, and monitors in real time while automatically identifying the signal. If an abnormal electrical signal that potentially causes the symptom is detected, the electrical stimulation for intervention is automatically applied, thereby forming a complete closed-loop neuromodulation system, effectively improving the effectiveness of treatment.

Second, the closed-loop neuromodulation system based on sacral nerve electrophysiological activity monitoring in the embodiments of the present disclosure stores the monitoring data via a server module, and trains the configured classifier based on the received local field potential variation signal, then sends the constructed intelligent recognition model after iteration to the programming module, which can determine in real time whether an abnormality in the current nerve signal exists, improving the efficiency and accuracy of recognition.

Third, the closed-loop neuromodulation system based on sacral nerve electrophysiological activity monitoring in the embodiments of the present disclosure better helps researchers or physicians to explore the correlation between the specificity of the local field potential change of the sacral nerve and the symptomatic phase of the patient by setting up the display unit, realizing on-demand acquisition, and quantitative and precise stimulation based on the individual bioelectrical signal indicators.

The foregoing is merely an example of an embodiment of the present disclosure and is not intended to limit the present disclosure. The present disclosure is subject to various changes and variations for those skilled in the art. Any modifications, equivalent substitutions, improvements, etc., which are made within the spirit and principles of the present disclosure shall be included in the scope of the claims of the present disclosure.

It should be noted that, in the present disclosure, unless otherwise expressly provided and limited, the terms “connection”, “fixed” and other terms should be understood in a broad sense. For example, it can be a fixed connection, a removable connection, or into a single unit; it can be a mechanical connection, an electrical connection or be communicable with each other; it can be a direct connection or an indirect connection through an intermediate medium, and it can be a connectivity within the two elements or an interactive relationship of the two elements unless otherwise expressly limited. For those skilled in the art, the specific meanings of the above terms in the context of the present disclosure may be understood on a case-by-case basis.

It should also be noted that the terms “includes”, “comprises”, or any other variant thereof, are intended to encompass non-exclusive inclusion, so that a process, method, commodity or apparatus comprising a set of elements includes not only those elements, but also other elements that are not explicitly listed, or that are inherent to such process, method, commodity or apparatus. In the absence of further limitation, the elements qualified by the statement “including a . . . ” do not preclude the existence of another identical element in the process, method, commodity, or apparatus that includes said element.

Moreover, certain terminology has been used to describe embodiments of the present disclosure. For example, the terms “one embodiment,” “an embodiment,” and/or “some embodiments” mean that a particular feature, structure, or feature described in connection with the embodiment is included in at least one embodiment of the present disclosure. Therefore, it is emphasized and should be appreciated that two or more references to “an embodiment”, “one embodiment”, or “an alternative embodiment” in various portions of the present disclosure are not necessarily all referring to the same embodiment. In addition, some features, structures, or characteristics of one or more embodiments in the present disclosure may be properly combined.

Furthermore, the recited order of processing elements or sequences, or the use of numbers, letters, or other designations, therefore, is not intended to limit the claimed processes and methods to any order except as may be specified in the claims. Although the above disclosure discusses some embodiments of the invention currently considered useful by various examples, it should be understood that such details are for illustrative purposes only, and the additional claims are not limited to the disclosed embodiments. Instead, the claims are intended to cover all combinations of corrections and equivalents consistent with the substance and scope of the embodiments of the present disclosure. For example, although the implementation of various components described above may be embodied in a hardware device, it may also be implemented as a software only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. However, this disclosure does not mean that the object of the present disclosure requires more features than the features mentioned in the claims. Rather, claimed subject matter may lie in less than all features of a single foregoing disclosed embodiment.

In some embodiments, the numbers expressing quantities or properties used to describe and claim certain embodiments of the present disclosure are to be understood as being modified in some instances by the term “about”, “approximate”, or “substantially”. For example, “about”, “approximate”, or “substantially” may indicate ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes. History application documents that are inconsistent or conflicting with the contents of the present disclosure are excluded, as well as documents (currently or subsequently appended to the present specification) limiting the broadest scope of the claims of the present disclosure. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the present disclosure disclosed herein are illustrative of the principles of the embodiments of the present disclosure. Other modifications that may be employed may be within the scope of the present disclosure. Thus, by way of example, but not of limitation, alternative configurations of the embodiments of the present disclosure may be utilized in accordance with the teachings herein. Accordingly, embodiments of the present disclosure are not limited to that precisely as shown and described.

What is claimed is:

1. A closed-loop neuromodulation system based on sacral nerve electrophysiological activity monitoring, wherein the closed-loop neuromodulation system comprises an implantable neuromodulation module, a programming module, and a server module, the implantable neuromodulation module is connected with the programming module, and the server module is connected with the programming module,
the implantable neuromodulation module is configured to:
obtain monitoring data generated during a sacral nerve conduction based on an implantable electrode and synchronously obtain a local field potential variation signal generated in a patient with urinary tract dysfunction during the sacral nerve conduction, the local field potential variation signal is caused by a neural activity that leads to urinary tract dysfunction;
apply an electrical stimulation pulse to a sacral nerve of the patient when an abnormal monitoring result of nerve afferent impulses associated with urinary urgency symptom of the patient is obtained based on the local field potential variation signal, wherein a control signal of the electrical stimulation pulse is generated by the programming module; and
when recognizing that the abnormal monitoring result returns to a normal monitoring result during an asymptomatic phase of the patient, deactivate, in response to recognizing the normal monitoring result, the electrical stimulation pulse applied to the sacral nerve after a preset delay time; and
the server module is configured to:
store the monitoring data and send an intelligent recognition model that is pre-trained to the implantable neuromodulation module and the programming module, respectively, wherein
the implantable neuromodulation module includes an intelligent recognition unit, and
the intelligent recognition unit is configured to:
identify, in real time, based on the intelligent recognition model, a first feature parameter corresponding to the local field potential variation signal generated by the patient with urinary tract dysfunction during the sacral nerve conduction, wherein the first feature parameter includes at least one of a potential intensity variation or a potential frequency variation; and
determine a first monitoring result corresponding to the local field potential variation signal based on the first feature parameter, and initiate the electrical stimulation pulse to the sacral nerve when the abnormal monitoring result of the nerve afferent impulses associated with the urinary urgency symptom of the patient exists.

2. The closed-loop neuromodulation system of claim 1, wherein the implantable neuromodulation module is operable offline or in conjunction with the programming module.

3. The closed-loop neuromodulation system of claim 1, wherein the implantable neuromodulation module is further configured to:
determine whether the abnormal monitoring result of the nerve afferent impulses associated with the urinary urgency symptom of the patient exists based on a first processing result of the local field potential variation signal and the intelligent recognition model, and send the local field potential variation signal obtained from monitoring to the programming module; and
the programming module is further configured to:
process the local field potential variation signal, and determine whether the abnormal monitoring result of the nerve afferent impulses associated with the urinary urgency symptom of the patient exists based on a second processing result of the local field potential variation signal and the intelligent recognition model; and
when the abnormal monitoring result of the nerve afferent impulses associated with the urinary urgency symptom of the patient exists, display interactive interface information and receive an electrical stimulation pulse parameter input from a physician to cause the implantable neuromodulation module to apply the electrical stimulation pulse to the sacral nerve.

4. The closed-loop neuromodulation system of claim 3, wherein the implantable neuromodulation module includes a first feature extraction unit configured to:
perform time-frequency processing on a local field potential variation signal after first filtering processing to generate a two-dimensional feature matrix of time and frequency, extract the first feature parameter, and send the first feature parameter to the intelligent recognition unit.

5. The closed-loop neuromodulation system of claim 4, wherein the programming module includes a display unit configured to:
display the interactive interface information, wherein the interactive interface information includes at least one of the local field potential variation signal transmitted by the implantable neuromodulation module, the second processing result and a second monitoring result of the local field potential variation signal obtained by the programming module, and a status interface of the implantable neuromodulation module.

6. The closed-loop neuromodulation system of claim 5, wherein the programming module further includes a parameter configuration unit configured to:
configure the electrical stimulation pulse parameter based on the interactive interface information of the display unit and send the electrical stimulation pulse parameter to a neurostimulation unit of the implantable neuromodulation module, to apply the electrical stimulation pulse to the sacral nerve via an electrode channel.

7. The closed-loop neuromodulation system of claim 3, wherein the programming module includes a second feature extraction unit and an algorithm configuration unit,
the second feature extraction unit is configured to:
perform time-frequency processing on a local field potential variation signal after second filtering processing to generate a two-dimensional feature matrix of time and frequency, extract a second feature parameter, and send the second feature parameter to the algorithm configuration unit; and
the algorithm configuration unit is configured to:
identify, in real time, based on the intelligent recognition model, the second feature parameter corresponding to the local field potential variation signal generated by the patient with urinary tract dysfunction during the sacral nerve conduction, wherein the second feature parameter includes at least one of a potential intensity variation or a potential frequency variation; and
determine a second monitoring result corresponding to the local field potential variation signal based on the second feature parameter, adjust a model parameter of the intelligent recognition model, and send the adjusted intelligent recognition model to the implantable neuromodulation module.

8. The closed-loop neuromodulation system of claim 1, wherein the server module is further configured to:
construct the intelligent recognition model, wherein the intelligent recognition model is obtained by training using a plurality of sets of training data through a machine learning algorithm, each set of training data among the plurality of sets of training data includes bioelectrical signals generated near the sacral nerve of the patient with urinary tract dysfunction during a symptomatic phase and the asymptomatic phase.

9. The closed-loop neuromodulation system of claim 1, wherein the implantable neuromodulation module further includes a sacral nerve signal acquisition unit configured to:
acquire, in real time, the local field potential variation signal generated by the patient with urinary tract dysfunction during the sacral nerve conduction, and send the local field potential variation signal to a first signal processing unit of the implantable neuromodulation module and a second signal processing unit of the programming module, respectively.

10. The closed-loop neuromodulation system of claim 6, wherein the electrical stimulation pulse parameter contained in the parameter configuration unit includes at least one of an amplitude, a pulse width, a frequency, a treatment duration, a variable/constant frequency, a high frequency, a low frequency, or a voltage/current source.
